(19) United States
(12) Patent Application Publication
(10) Pub. No.: US 2005/0009154 A1
(43) Pub. Date: Jan. 13, 2005

(54) Biocatalytic preparation of 1-cyano-cyclohexaneacetic acid

(75) Inventors: John Wing Wong, East Lyme, CT (US); Michael Paul Burns, Mystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/869,518

(22) Filed: Jun. 16, 2004

Related U.S. Application Data
(60) Provisional application No. 60/479,659, filed on Jun. 19, 2003.

(51) Int. Cl. C12P 13/00

(52) U.S. Cl. 435/128

(57) ABSTRACT

The present invention is directed to novel biocatalytic processes for the conversion of aliphatic ∝, ω-dinitriles into the corresponding ω-nitrilecarboxylic acid. More particularly, the present invention provides a method for the conversion of 1-cyanocyclohexaneacetonitrile to 1-cyanocyclohexaneacetic acid using an enzyme catalyst with nitrilase activity. 1-cyanocyclohexaneacetic acid is a useful intermediate in the synthesis of gabapentin.

### FIELD OF THE INVENTION

The present invention is directed to novel biocatalytic processes for the conversion of an aliphatic ∝, ω-dinitrile into the corresponding ω-nitrilecarboxylic acid. More particularly, the present invention provides methods for the conversion of 1-cyanocyclohexaneacetonitrile to 1-cyanocyclohexaneacetic acid, which is a useful intermediate in the synthesis of gabapentin. Gabapentin can be used for the therapy of certain cerebral diseases, for example, certain forms of epilepsy, faintness attacks, hypokinesia and cranial traumas. Since gabapentin is effective in improving cerebral functions, it is also useful in the treatment of geriatric patients.

### BACKGROUND OF THE INVENTION

The use of a nitrilase enzyme to prepare a carboxylic acid from the corresponding nitrile is disclosed in WO 02/072856. Incorporation of the enzyme into a polymer matrix with cross-linking provided a catalyst with improved physical and biochemical integrity.

The regioselective preparation of ω-nitrilecarboxylic acids from aliphatic ∝, ω-dinitriles with a biocatalyst was disclosed in U.S. Pat. No. 5,814,508 ('508 patent). For example, a catalyst having nitrilase activity was used to convert 2-methylglutaronitrile into 4-cyanopentanoic acid.

K. Yamamoto, et al. J. Ferment. Bioengineering, 1992, vol. 73, 125-129 describes the use of microbial cells having both nitrile hydratase and amidase activity to convert trans 1,4-dicyanocyclohexane to trans-4-cyanocyclohexanecarboxylic acid.

Regioselective biocatalytic conversions of dinitriles to cyano substituted carboxylic acids, have been reported for a series of aliphatic α, ω-dinitrile compounds using microbial cells having an aliphatic nitrilase activity or a combination of nitrile hydratase and amidase activities (J. E. Gavagan et al. J. Org. Chem., 1998, vol. 63, 4792-4801). The foregoing references are hereby incorporated in their entirety.

Generally, enzyme-catalyzed conversions of nitriles to the corresponding carboxylic acids have advantages over chemical processes that use strongly acidic or basic conditions and high temperatures. In addition to operating under milder reaction conditions, the enzyme-catayzed conversion of dinitriles to nitrilecarboxylic acids occurs with high regioselectivity so that only one of two nitrile groups undergoes reaction.

### SUMMARY OF THE INVENTION

In the process of the present invention, regioselective -biocatalytic conversions of 1-cyanocyclohexaneacetonitrile to 1-cyanocyclohexaneacetic acid are achieved using enzyme catalysts having aliphatic nitrilase activity.

The present invention comprises a process for preparing 1-cyanocyclohexaneacetic acid from 1-cyanocyclohexaneacetonitrile comprising the steps of
(a) contacting 1-cyanocyclohexaneacetonitrile with an enzyme catalyst having nitrilase activity in an aqueous reaction mixture; and
(b) recovering 1-cyanocyclohexaneacetic acid from the aqueous reaction mixture.

Whole microbial cell enzyme catalysts that have aliphatic nitrilase activity and are useful in the present invention include Acidovorax facilis 72W (ATCC 55746), Acidovorax facilis 72-PF-15 (ATCC 55747), Acidovorax facilis 72-PF-17 (ATCC 55745), Escherichia coli SS1001 (ATCC PTA-1177) Escherichia coli SW91 (ATCC PTA-1175) and Bacillus sphaericus (ATCC).

Preferably, the enzyme catalyst is selected from the group consisting of Acidovorax facilis 72W (ATCC 55746), Escherichia coli SS/001 (ATCC PTA-177) and Escherichia coli SW 91 (ATCC PTA 1175).

In another embodiment, the enzyme catalysts are immobilized in a polymer matrix. Preferably the polymer matrix is calcium alginate.

Preparations of partially purified enzymes that have aliphatic nitrilase activity and are useful for the conversion of II into I include NIT-104, NIT-105, and NIT-106 (Biocatalytics Inc., Pasadena, Calif.).

In a preferred embodiment, the step of contacting 1-cyanocyclohexaneacetonitrile with an enzyme catalyst involves the step of pre-dissolving the 1-cyanocyclohexaneacetonitrile in a water miscible organic solvent. Most preferably the solvent is dimethyl formamide (DMF) or dimethylsulfoxide (DMSO).

In another embodiment of the present invention, the 1-cyanocyclohexaneacetic acid is recovered from the aqueous reaction mixture by extraction with an organic solvent. Preferably the organic solvent used in the extraction step is ethyl acetate or methyl tertiary butyl ether.

### DETAILED DESCRIPTION OF THE INVENTION

Those skilled in the art will fully understand the terms used herein to describe the present invention; nonetheless, the following terms or abbreviations used herein, are as described immediately below.
“° C” means degrees-Celsius;
“Enzyme catalyst” means a catalyst which is characterized by either a nitrilase activity or a combination of a nitrile hydratase activity and an -amidase activity. The catalyst may be in the form of a whole microbial cell, permeabilized microbial cell(s), one or more cell component of a microbial cell extract, partially purified enzyme(s), or purified enzyme(s);
“Aqueous reaction mixture” means a mixture of the substrate and enzyme catalyst in a largely aqueous medium;
“Nitrilase activity” means an enzyme activity that converts a nitrile group to a carboxylic acid group;
“Nitrile hydratase activity” means an enzyme activity that converts a nitrile group to an amide group;
“Amidase activity” means an enzyme activity that converts an amide group to a carboxylic acid group;

ATCC is American Type Culture Collection located at 10801 University Boulevard, Manassas, Va., 20110-2209, U.S.A. Biocatalytics Inc. is located at 129 N. Hill Avenue, Suite 103, Pasadena, Calif., 91106, U.S.A. Zylepsis Ltd. Is located at Henwood Business Estate, Ashford, Kent, U.K. TN24 8DH.

The present invention provides a biocatalytic method for preparing 1-cyanocyclohexaneacetic acid (I) from 1-cyanocyclohexaneacetonitrile (II) as follows:

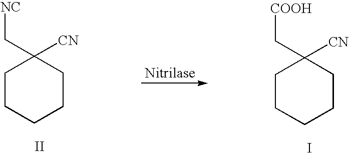

This biocatalytic process is carried out by contacting the compound of Formula II with an enzyme catalyst having nitrilase activity, and produces the compound of Formula I in high yields and high regioselectivity.

This biocatalytic process can also be carried out by contacting the compound of Formula II with an enzyme catalyst having a combination of nitrile hydratase and amidase activities. Whereas contacting the compound of Formula II with an enzyme catalyst having nitrilase activity results in the formation of I in a single step, formation of I using an enzyme catalyst having nitrile hydratase and amidase activities involves the formation of 2-(1-cyano-cyclohexyl)-acetamide by contact of II with the nitrile hydratase activity followed by hydrolysis of 2-(1-cyano-cyclohexyl)-acetamide to I by the amidase activity. Zyanotase™ (Zylepsis Ltd., Ashford, Kent, U.K) is a suitable enzyme catalyst for the conversion of 1-cyanocyclohexaneacetonitrile to 2-(1-cyano-cyclohexyl)-acetamide.

Various enzymes of the present invention, having nitrilase activity or a combination of nitrile hydratase and amidase activities, can be found through screening protocols such as enrichment isolation techniques, which initially select microorganisms based on their ability to grow in media containing the enrichment nitrile. Enrichment isolation techniques typically involve the use of carbon-limited or nitrogen-limited media supplemented with an enrichment nitrile, which can be the nitrile substrate for the desired bioconversion, or a structurally similar nitrile compound. Microorganisms that possess nitrilase activity can be initially selected based on their ability to grow in media containing the enrichment nitrile. Gavagan et al., (Appl. Microbiol. Biotechnol. (1999) vol. 52, 654-659) used enrichment techniques to isolate a Gram-negative bacterium, Acidovorax facilis 72W (ATCC 55746), from soil, using 2-ethylsuccinonitrile as the sole nitrogen source. Acidovorax facilis 72W (ATCC 55746) was shown to be useful for the selective conversion of 2-methylglutaronitrile to 4-cyanopentanoic acid. Enrichment techniques were also used to isolate the thermophilic bacterium, Bacillus pallidus Dac521, which catalyzes the conversion of 3-cyanopyridine to nicotinic acid (Almatawah and Cowan, Enzyme Microb. Technol. (1999) vol. 25, 718-724). Microorganisms isolated by enrichment techniques can be tested for nitrile hydrolysis activity by contacting suspensions of microbial cells with a nitrile compound and testing for the presence of the corresponding carboxylic acid using analytical methods such as high performance liquid chromatography, gas liquid chromatography, or liquid chromatography mass spectrometry (LCMS). Techniques for testing the nitrile hydrolysis activity of Acidovorax facilis 72W (ATCC 55746) are reported in U.S. Pat. No. 5,814,508. Enrichment techniques were used to isolate one microorganism from soil, which could grow on 1-cyanocyclohexaneacetonitrile as a nitrogen source. This microorganism, identified as Bacillus sphaericus (ATCC ______) using a Vitek metabolic assay, was shown to convert II to I.

Once a microorganism having nitrilase activity or nitrile hydratase and amidase activities has been isolated, enzyme engineering can be employed to improve various aspects of the enzyme(s). These improvements can be useful for the present invention and include increasing catalytic efficiency of the enzyme, increasing stability to higher temperatures, a wider range of pH, and enabling the enzyme to operate in a reaction medium including a mixture of aqueous buffer and organic solvent.

A variety of techniques, which can be employed in the present invention, to produce an enzyme catalyst having nitrilase activity or nitrile hydratase and amidase activities in addition to having an improved yield, throughput, and product quality suitable for a particular bioconversion process, include but are not limited to enzyme engineering techniques such as rational design methods such as site-directed mutagenesis and directed evolution techniques utilizing random mutagenesis or DNA shuffling techniques.

Suitable enzyme catalysts for the conversion of II into I are in the form of whole microbial cells, permeabilized microbial cells, extracts of microbial cells, partially purified enzymes or purified enzymes, and such catalysts can be immobilized on a support.

This process can be carried out by contacting 1-cyanocyclohexaneacetonitrile with an enzyme catalyst in distilled water, or in an aqueous solution of a buffer, which will maintain the initial pH of the reaction between 5.0 and 10.0, preferably between 6.0 and 8.0. Suitable buffering agents include potassium phosphate and calcium acetate. As the reaction proceeds, the pH of the reaction mixture may change due to the formation of an ammonium salt of the carboxylic acid from the corresponding nitrile functionality of the dinitrile. The reaction can be run to complete conversion of dinitrile with no pH control, or a suitable acid or base can be added over the course of the reaction to maintain the desired pH. However, as indicated above it is possible to produce enzyme catalysts using technologies such as enzyme engineering and directed evolution, which will operate effectively over wider pH ranges.

In one particular embodiment, whole microbial cells are used as catalysts. The whole microbial cells can be used without pretreatment; however, Acidovorax facilis cells are preferably heat treated at about 50° C. for about 1 hour which results in the deactivation of an undesirable nitrile hydratase activity and produces a whole cell catalyst that is highly regioselective for the conversion of II to I. Acidovorax facilis 72-PF-15 (ATCC 55747) and Acidovorax facilis 72-PF-17 (ATCC 55745) alternatively, produce very low levels of the undesireable nitrile hydratase activity and thus do not require heat treatment before use as an enzyme catalyst for the conversion of II to I. The wet cell weight of the microbial whole cell enzyme catalyst typically ranges from about 0.001 g/mL to about 0.5 g/mL and preferably from about 0.1 g/mL to about 0.3 g/mL.

Optionally, the catalyst may be immobilized in a polymer matrix. Immobilized enzyme catalysts can be used repeatedly and in continuous processes, and can be separated from the products of the enzymatic process more easily than un-immobilized enzyme catalysts. Particularly, in the present invention, whole cells can be immobilized by entrapment in a polymer matrix such as calcium alginate or polyacrylamide. Inorganic solid supports such as celite are also used. Methods for the immobilization of cells in a polymer matrix are well-known to those skilled in the art. Immobilized cells of Acidovorax facilis 72W (ATCC 55746), Escherichia coli SW91 (ATCC PTA-1175), and Escherichia coli SS1001 (ATCC PTA-1177) are particularly useful for the conversion of II to I, since they can be used repeatedly in batch processes or in continuous processes. Cells of Acidovorax facilis 72W (ATCC 55746), Escherichia coli SW91 (ATCC PTA-1175), and Escherichia coli SS1001 (ATCC PTA-1177), immobilized in calcium alginate or carrageenan (WO 01/75077 A2) are useful for the conversion of II to I. Preferably, the enzyme catalyst consisting of whole cells entrapped in a polymer matrix is used in the range of about 0.01 g to 0.6 g wet weight per mL of reaction volume, with a preferred range of 0.1 to 0.5 g/mL

Additionally, several lyophilized lysates prepared from microbial cells and designated as NIT-104, NIT-105, and NIT-106 (Biocatalytics Inc., Pasadena, Calif.), are also useful for the conversion of II to I. Contact of NIT-104, NIT-105, and NIT-106 with II in an aqueous reaction mixture results in the formation of I. Substrate and catalyst concentrations of 0.01 to 10 g/L can be used, with a preferred range of 0.1 to 5 g/L. Reaction conditions (temperature and pH ranges) described for whole cell and immobilized whole cell enzyme catalysts can also be used for the conversion of II to I using lyophilized lysates.

The temperature of the hydrolysis reaction is chosen to both optimize both the reaction rate and the stability of the enzyme catalyst activity. The temperature of the reaction may range from just above the freezing point of the suspension (ca. 0° C.) to 60° C., with a preferred range of reaction temperature of from 5° C. to 35° C.

The enzyme-catalyzed conversion of II to I can be carried out by contacting II with the enzyme catalyst in an aqueous reaction mixture. Compound II, the starting material, which is only moderately water soluble, (ca. 10 mM, 25° C., 20 mM phosphate buffer, pH 7), can be added to an aqueous reaction containing the enzyme catalyst at levels exceeding its aqueous solubility limit. Thus reaction mixtures initially consist of two phases, an aqueous phase containing dissolved II and the enzyme catalyst, and a solid phase containing undissolved II. At complete conversion of II, a single phase containing compound I and the enzyme catalyst remains. The enzyme catalyzed conversion of II to I can be carried out with levels of compound II from about 0.1 g/L to 148 g/L, with a preferred range of about 0.1 g/L to 90 g/L. The enzyme catalyst concentrations used in the present invention depend on the specific activity of the enzyme catalyst and is chosen to obtain the desired rate of reaction.

Subsequent to the conversion the reaction product isolation by extraction with an organic solvent, such as ethyl acetate or methyl tertiary butyl ether, is preferred. Yields of 1-cyanocyclohexaneacetic acid range from about 29% to about 97%.

As is well known to those skilled in the art, a variety of methods may be used to recover the carboxylic acid of Formula I.

The compound of Formula I, 1-Cyanocyclohexaneacetic acid, produced by the processes of the present invention can be further reacted to produce 1-aminomethyl-1-cyclohexaneacetic acid (gabapentin, compound of Formula III), as described in Example 9 of the present invention and disclosed in U.S. Pat. No. 5,362,883.

The catalytic hydrogenation of a salt or ester of 1- cyanocyclohexaneacetic acid (Ia) into gabapentin (III) is carried out as follows: 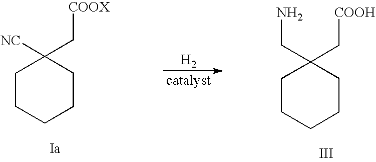
wherein X is an alkali metal or an alkaline earth metal or C₁-C₆ alkyl.

Alternative synthetic methods for the preparation of gabapentin, the compound of Formula III include (a) converting a monoalkyl ester of 1,1-cyclohexane-diacetic acid into an azide which is subjected to the Curtius rearrangement, and (b) subjecting 1,1-cyclohexane-diacetic acid monoamide to the Hofmann rearrangement as disclosed in U.S. Pat. No. 4,024,175.

In another process for the preparation of the compound of Formula III, gabapentin, as disclosed in U.S. Pat. No. 5,693,845, 1-cyanocyclohexaneacetonitrile is converted into the corresponding cyano imidoester in situ which upon hydrolysis and hydrogenation affords gabapentin.

Gabapentin is a useful drug in the treatment of a variety of central nervous system disorders including certain psychiatric and neurological diseases. Gabapentin exhibits anticonvulsant and antispastic activity with an extremely low toxicity in man. Additionally, gabapentin has found wide use for chronic pain and for general improvements in cerebral functions making it a drug of choice in the treatment of geriatric patients (M. P. Davis and M. Srivastava, Drugs & Aging, 2003, 001.20, 23-57).

The compounds of formula III can be administered enterally or parenterally within wide dosage ranges in liquid or solid form. As injection solution, water is preferably employed which contains the usual additives for injection solutions, such as stabilising agents, solubilising agents and/or buffers.

Additives of this kind include, for example, tartrate and citrate buffers, ethanol, complex-forming agents (such as ethylenediamine-tetraacetic acid and the non-toxic salts thereof), as well as high molecular weight polymers (such as liquid polyethylene oxide) for viscosity regulation. Solid carrier materials include, for example, starch, lactose, mannitol, methyl cellulose, talc highly-dispersed silicic acids, high molecular weight fatty acids (such as stearic acid), gelatine, agar-agar, calcium phosphate, magnesium stearate, animal and vegetable fats and solid high molecular weight polymers (such as polyethylene glycol); compositions suitable for oral administration can, if desired, also contain flavouring and/or sweetening agents.

The individual dosage for gabapentin can be 5 mg-50 mg parenterally and 20 mg-200 mg enterally.

The following Examples are given for the purpose of illustrating the present invention:

### EXAMPLE 1

1-Cyanocyclohexaneacetic acid from 1-cyanocyclohexaneacetonitrile (NIT-104, NIT-105, and NIT-106)

1-Cyanocyclohexaneacetonitrile (5 mg in 0.05 mL dimethylsulfoxide) was added to each of three 8 mL screw cap glass vials containing 5 mg of NIT-104, NIT-105, or NIT-106 (Biocatalytics Inc., Pasadena, Calif.), and 1 mL of 50 mM potassium phosphate buffer (pH 7.5, 2 mM dithiothreitol (DTT)). The resulting mixtures were stirred at 21° C. using a magnetic stirrer. After 24 hour, samples were withdrawn from each reaction mixture and analyzed by Liquid Chromatography/Mass Spectroscopy (LCMS). The LCMS analyses indicated 100% yields of 1-cyanocyclohexaneacetic acid using NIT104, NIT-105, and NIT-106.

### EXAMPLE 2

1-Cyanocyclohexaneacetic acid from 1-cyanocyclohexaneacetonitrile (NIT-104)

1-Cyanocyclohexaneacetonitrile (25 mg in 0.25 mL of DMF) was added to an 8 mL screw cap glass vial containing 25 mg of NIT-104 in 5 mL of 50 mM potassium phosphate buffer (pH 7.5, 2 mM DTT), and stirred at 21° C. for 24 hour. The reaction mixture was then extracted with two 7 mL portions of ethyl acetate, which were discarded. The aqueous layer was acidified to pH 2 with 4N HCl, and extracted with three 7 mL portions of ethyl acetate. The ethyl acetate extracts were combined, dried over anhydrous magnesium sulfate, and concentrated under vacuum to give 14 mg of 1-cyanocyclohexaneacetic acid (50% yield).

### EXAMPLE 3

1-Cyanocyclohexaneacetic acid from 1-cyanocyclohexaneacetonitrile (Acidovorax facilis 72W ATCC 55746)

The bioconversion of 1-cyanocyclohexaneacetonitrile to 1-cyanocyclohexaneacetic acid was carried out with cells of Acidovorax facilis 72W (ATCC 55746) prepared using procedures similar to those described in U.S. Pat. No. 5,858,736 and International Patent Application WO 01/75077 A2 incorporated by reference herein. Specifically, a Tryptic soy agar plate was inoculated with cells of A. facilis 72W and incubated overnight at 29° C. Three 300 mL Erlenmeyer flasks, each containing 25 mL of medium A (potassium phosphate, monobasic, 0.39 g/L; potassium phosphate, dibasic, 0.39 g/L; Difco yeast extract, 5.0 g/L; pH 6.9), were inoculated with A. facilis 72W cells from the agar plate and incubated on a rotary shaker (230 rpm) overnight at 27° C. The contents of the three flasks were pooled and used to inoculate ten 300 mL Erlenmeyer flasks, each containing 25 mL of medium A (1.5 mL inoculum per flask), and sixteen 500 mL Erlenmeyer flasks, each containing 35 mL of medium A (1.75 mL inoculum per flask). These flasks were incubated on a rotary shaker (230 rpm) at 27° C. for 48 h, after which the contents were combined, treated with glycerol (10% v/v), and centrifuged. The pellet was resuspended in 100 mL of 20 mM potassium phosphate (10% glycerol) and incubated at 50° C. for 50 minutes. After heat-treatment, the cells were recovered by centrifugation, frozen in dry ice, and stored at −80° C.

Two 1 gram (wet cell weight) aliquots of frozen, heat-treated A. facilis 72W cells were separately thawed in 12 mL of 100 mM potassium phosphate buffer (pH 7.0, buffer A), centrifuged, and resuspended in 20 mL of buffer A. The cell suspensions were transferred to two 100 mL jacketed reaction vessels (A and B) maintained at 30° C. 1-Cyanocyclohexaneacetonitrile (296 mg) was added to each reaction vessel. In the case of vessel A, the substrate was dissolved in 1 mL of DMSO, while for vessel B the substrate was added without solvent. The reactions were both stirred for 22 hour using stirring attachments provided with Graphix DL50 titrators (Mettler-Toledo, Columbus, Ohio). The reaction mixtures were each extracted twice with 20 mL aliquots of ethyl acetate, which were discarded. The aqueous layers were acidified to pH 2 with 4N HCl, and extracted with ethyl acetate (3×40 mL). The ethyl acetate extracts were then dried over anhydrous magnesium sulfate, filtered, and concentrated under vacuum. Yields of 1-cyanocyclohexaneacetic acid from reactions A and B were 324 mg (97%) and 273 mg (82%), respectively.

### EXAMPLE 4

1-Cyanocyclohexaneacetic acid from 1-cyanocyclohexaneacetonitrile (Acidovorax facilis 72W ATCC 55746)

Frozen, heat-treated A. facilis 72W cells (2 g wet cell weight) were prepared as described in Example 3, resuspended in 20 mL of buffer A, and transferred to a 100 mL jacketed reaction vessel maintained at 30° C. 1-Cyanocyclohexaneacetonitrile (1.48 g) was added to the cell suspension and the resulting mixture was stirred for 72 hour. The reaction mixture was centrifuged, and the pellet resuspended in 20 mL of 20 mM potassium phosphate buffer (pH 7) and centrifuged again. The supernatants from both centrifugations were combined and extracted with ethyl acetate as described in Example 3 to give 1.30 g (77.8% yield) of 1-cyanocyclohexaneacetic acid.

### EXAMPLE 5

1-Cyanocyclohexaneacetic acid from 1-cyanocyclohexaneacetonitrile (Acidovorax facilis 72W ATCC 55746)

Frozen, heat-treated A. facilis 72W cells were prepared using a procedure similar to that described in Example 3. Nine 300 mL Erlenmeyer flasks, each containing 30 mL of medium B (Difco yeast extract, 5 g/L; potassium phosphate, monobasic, 1.19 g/L; potassium phosphate, dibasic, 2.83 g/L, Nutrient Feed solution, 26 mL (International Patent Application WO 01/75077 A2) incorporated by reference, pH 7.0), were inoculated with A. facilis 72W cells and incubated on a rotary shaker (220 rpm) at 27° C. for 72 hour. The contents of each 300 mL flask were separately added to nine Fernbach flasks, each containing 300 mL of medium B. The Fernbach flasks were incubated on a rotary shaker (220 rpm) at 27° C. After 72 hour, the contents of the Fernbach flasks were centrifuged to a pellet, which was resuspended in 310 mL of 20 mM potassium phosphate (pH 7.0) and placed in a 50° C. water bath for 1 hour. The heat-treated cell suspension was centrifuged to a pellet, and then frozen in dry ice and stored at −80° C.

Frozen, heat-treated A. facilis 72W cells (27 g), prepared as described above, were resuspended in 100 mL of 100 mM phosphate buffer (pH 7.0) and added to a 100 mL jacketed reaction vessel containing 7.4 g of 1-cyanocyclohexaneacetonitrile. The resulting mixture was stirred at 30° C. for 23 hour. The reaction mixture was centrifuged, and the resulting pellet resuspended in 50 mL of 20 mM potassium phosphate buffer (pH 7) and centrifuged again. The supernatants were combined and extracted with 200 mL of ethyl acetate resulting in the formation of an emulsion. Phosphate buffer (200 mL, 0.5M, pH 7) and 100 mL water were added to the emulsion. The aqueous layer was separated, adjusted to pH 2 with 4N HCl, and extracted with ethyl acetate (3×500 mL). The combined ethyl acetate extracts were dried over anhydrous magnesium sulfate, filtered, and concentrated under vacuum to give 7.47 g (89.4% yield) of 1-cyanocyclohexaneacetic acid.

### EXAMPLE 6

1-Cyanocyclohexaneacetic acid from 1-cyanocyclohexaneacetonitrile (Calcium alginate-immobilized E. coli transformant SS1001)

1-Cyanocyclohexaneacetonitrile was converted to 1-cyanocyclohexaneacetic acid using calcium alginate-immobilized E. coli transformant SS1001 (DuPont, Wilmington, Del.). A 100 mL glass, jacketed reaction vessel, maintained at 30° C., was charged with 1.48 g 1-cyanocyclohexaneacetonitrile, 2 mL 50 mM calcium acetate buffer (pH 7.0), and water to bring the total weight of the reaction vessel's contents to 20 g. E. coli SS1001/alginate beads (10 g, International Patent Application WO 01/75077 A2) incorporated by reference were added to the reaction vessel and the resulting mixture stirred with a magnetic stir bar. After 7 hour, the product mixture was decanted, and the biocatalyst beads washed twice with 10 mL aliquots of 5 mM calcium acetate buffer (pH 7.0). Seventeen additional batch reactions were carried out as described above using the recycled biocatalyst beads. Approximately two reactions were carried out in a 24 hour period. Reactions started in the morning were decanted after 7 hour, while reactions started in the afternoon were allowed to run overnight and decanted after 16 hour. The decanted product mixtures and bead washings were combined and extracted with ethyl acetate (discarded). The aqueous layer was separated, acidified to pH 2 with 4N HCl, and extracted with ethyl acetate. The ethyl acetate extracts were dried over anhydrous magnesium sulfate, filtered, and concentrated under vacuum to give 26.4 g (87.8% yield) of 1-cyanocyclohexaneacetic acid. After the initial 17 consecutive batch reactions, the recycled biocatalyst beads were used for another 18 consecutive batch reactions. These reactions were carried out as described above, with 0.74 g substrate (1 batch, 7 hour reaction time), 1.48 g substrate (8 batches, 7 hour or 16 hour reaction time), or 2.22 g substrate (8 batches, 24-31 hour reaction times). The combined product mixtures were extracted with ethyl acetate as described above to give 31.3 g (91.4% yield) of 1-cyanocyclohexaneacetic acid.

### EXAMPLE 7

1-Cyanocyclohexaneacetic acid from 1-cyanocyclohexaneacetonitrile (Bacillus sphaericus (ATCC ______))

Bacillus sphaericus (ATCC ______) was isolated from soil collected in Groton, Conn. by standard enrichment techniques using a basal medium (KH₂PO₄ 1.5 g/L, KH₂PO₄ 3.4 g/L, KCl 0.5 g/L, NaCl 1.0 g/L, MgSO₄ 0.24 g/L, sodium citrate 0.2 g/L, HCl, 0.01 mL/L, CaCl₂.H₂O 0.11 g/L, MnSO₄.H₂O 0.01 g/L, CuSO₄.5H₂O 0.006 g/L, boric acid 0.009 g/L, ZnSO₄.7H₂O 0.018 g/L, NaMoO₄.2H₂O 0.0005 g/L, VnSO₄.H₂O 0.0008 g/L, NiNO₃.6H₂O 0.0004 g/L, Na₂Se 0.0004 g/L, FeSO₄.7H₂O 0.06 g/L, biotin 0.0002 g/L, folic acid 0.0002 g/L, pyridoxine.HCl 0.001 g/L, riboflavin 0.0005 g/L, thiamine.HCl 0.00005 g/L, nicotinic acid 0.0005 g/L, pantothenic acid 0.0005 g/L, vitamin B12 0.00001 g/L, p-aminobenzoic acid 0.0005 g/L.) supplemented with 0.2% 1-cyanocyclohexaneacetonitrile. Bacillus sphaericus (ATCC ______) was selected based on growth in the supplemented basal medium and isolated by repeated passages on agar plates of the same medium. Selected colonies were grown on Brain Heart Infusion agar to ensure purity. Bacillus sphaericus (ATCC ______) grew as round, glossy, orange colonies of 1-2 mm on Brain Heart Infusion agar plates and was identified using a Vitek metabolic assay.

Cells of Bacillus sphaericus (ATCC ______) were grown in shake flask cultures (300 ml flasks , 35 ml medium) on basal medium supplemented with 0.5% yeast extract. After 18 h at 29° C., cells were harvested by centrifugation, washed with 20 mM potassium phosphate (pH 7.0) and resuspended to 50 mg/mL in the same buffer. 1-Cyanocyclohexaneacetonitrile was added to the suspension of cells at a concentration of 1.48 g/L and shaken for five days at 26° C. The aqueous reaction mixture was then extracted with ethyl acetate and analyzed by LCMS to reveal a 29% yield of 1-cyanocyclohexaneacetic acid.

### EXAMPLE 8

2-(1-cyano-cyclohexyl)-acetamide from 1-cyanocyclohexaneacetonitrile (Zyanotase)

To a 125 mL round bottom flask was added 1-cyanocyclohexaneacetonitrile (0.59 g, 4 mmol), Zyanotase™ (120 mg, Zylepsis Ltd), and 40 mL of potassium phosphate (100 mM, pH 7). The reaction mixture was stirred at 21° C. for 48 h and then extracted with two 40 mL aliquots of ethyl acetate. The combined ethyl acetate extracts were concentrated on a rotary evaporator to give 550 mg (82.7% yield) of 2-(1-cyano-cyclohexyl)-acetamide.

### EXAMPLE 9

1-Aminomethyl-cyclohexaneacetic acid from 1-cyanocyclohexaneacetic acid (U.S. Pat. No. 5,362,883)

To a 500-mL Parr bomb is added 23.5 g (0.1 mol) of 1-cyanocyclohexaneacetic acid, 28% water wet; 16 g of 50% water wet Raney nickel #30, and a cooled (20° C.) methyl alcohol (160 mL) and 50% aqueous sodium hydroxide (8.8 g, 0.11 mol) solution. The reaction mixture is stirred at 22° C. to 25° C. for 21 hours at 180 pounds per square inch gauge (psig) hydrogen. After 21 hours, the hydrogen is vented and the reduced mixture is flushed with nitrogen.

The reaction mixture is pressure filtered over celite, washed with methyl alcohol (100 mL), and stripped to a volume of 50 mL at 35° C. on the rotary evaporator. Isopropyl alcohol (100 mL) is added followed by the dropwise addition of 6.6 g (0.11 mol) of acetic acid. The product solution is stripped on the rotary evaporator to a volume of 50 mL. Tetrahydrofuran (125 mL) is added to the concentrated product solution, the solution cooled in an ice bath, suction filtered, and washed using 50 mL of tetrahydrofuran. The crude product cake is dried under vacuum at 45° C. for 16 hours.

The crude product is recrystallized from methyl alcohol, demineralized water, and isopopyl alcohol to yield 10.3 g of 1-(aminomethyl)-cyclohexaneacetic acid as a crystalline white solid. The high-performance liquid chromatography (HPLC) results show no organic impurities detected with a 97.2% weight/weight (w/w) purity.

1. A process for preparing 1-cyanocyclohexaneacetic acid from 1-cyanocyclohexaneacetonitrile comprising the steps of
(a) contacting 1-cyanocyclohexaneacetonitrile with an enzyme catalyst having nitrilase activity in an aqueous reaction mixture; and
(b) recovering 1-cyanocyclohexaneacetic acid from the aqueous reaction mixture.

2. The method of claim 1 wherein said enzyme catalyst in the form of whole microbial cells is selected from the group of cells consisting of Acidovorax facilis 72W (ATCC 55746), Acidovorax facilis 72-PF-15 (ATCC 55747), Acidovorax facilis 72-PF-17 (ATCC 55745), Escherichia Coli SS1001 (ATCC PTA-1177) Escherichia coli SW91 (ATCC PTA-1175) and Bacillus sphaericus (ATCC).

3. The method of claim 2 wherein said enzyme catalyst is selected from the group consisting of Acidovorax facilis 72W (ATCC 55746), Escherichia Coli SS1001 (ATCC PTA 1177) and Escherichia coli SW91 (ATCC PTA 1175).

4. The method of claim 1 wherein said enzyme catalyst is a partially purified enzyme selected from the group consisting of NIT-104, NIT-105, and NIT-106.

5. The method of claim 2 wherein said enzyme catalyst is immobilized in a polymer matrix.

6. The method of claim 5 wherein said enzyme catalyst is immobilized in calcium alginate.

7. A process according to claim 1 wherein the 1-cyanocyclohexaneacetonitrile in (a) is pre-dissolved in a water miscible inert organic solvent.

8. The process according to claim 1 wherein said recovery step in (b) comprises extracting the aqueous reaction mixture with an organic solvent.

9. The process according to claim 8 wherein said organic solvent is ethyl acetate or methyl tertiary butyl ether.

10. The process according to claim 7 wherein said water miscible organic solvent is DMF or DMSO.
